(19) United States
(12) Patent Application Publication
(10) Pub. No.: US 2017/0173066 A1
(43) Pub. Date: Jun. 22, 2017

(54) METHOD OF FEEDING ANIMALS GLUCOMANNOPROTEIN PRODUCTS

(71) Applicant: PURINA ANIMAL NUTRITION LLC, Shoreview, MN (US)

(72) Inventor: Bill L. Miller, Labadie, MO (US)

(21) Appl. No.: 14/978,780

(22) Filed: Dec. 22, 2015

(51) Int. Cl. A61K 31/715; A23K 50/10; A61K 45/06; A23K 10/30; A23K 20/195; A61K 9/00; A23K 20/147; A23K 20/158

(52) U.S. Cl. CPC A61K 31/715; A23K 20/147; A23K 50/10; A23K 20/158; A23K 10/30; A23K 20/195; A61K 9/0056; A61K 45/06

(57) ABSTRACT

Methods involve feeding ruminants a milk replacer, where the milk replacer comprises an amount of soluble mannan oligosaccharides. The ruminants may be calves, and may be between about 0 and about 6 weeks of age. In response to ingesting the milk replacer, the ruminants may increase a rate of weight gain.

### TECHNICAL FIELD

Implementations relate to ruminant feed products and methods of feeding such ruminant feed products. More particularly, implementations provide methods of feeding milk replacers with soluble mannan oligosaccharides to ruminants resulting in improved animal performance.

### BACKGROUND

Ruminant animals require adequate nutrition for healthy growth and development. Robust growth is especially important for certain classes of ruminants, such as livestock, which are a commodity raised to produce food products including milk and meat. The time it takes livestock to mature, particularly to gain weight, is important when assessing whether the animal is ready to produce milk or is ready to go to market. A number of feeding systems have been used to enhance weight gain of livestock beginning at a young age and may include feeding techniques prior to and after weaning. These techniques may involve providing milk replacer products to the animals. Milk replacers generally mimic the milk produced from the post-partum parent animal in terms of protein, fat and carbohydrate content. Providing nutrients to young animals through milk replacers may, for instance, reduce the age of freshening or the onset of lactation of a dairy cow, thereby reducing the cost of milk production. Increased weight gain of livestock from an early age by ingestion of milk replacer may also reduce the cost of beef production.

A concern for producers is whether ruminant animals are receiving adequate nutrients. Because the effects of poor nutrition can be exacerbated at a young age, ensuring that young ruminants are provided with the necessary nutritive sources is especially important. Differences in feed intake by animals impact the rate of weight gain and ultimately body size. Additionally, the ingestion of adequate nutrients does not guarantee that ruminant animals will ultimately receive the benefit of these nutrients, as harmful bacteria residing within the gastrointestinal tract can prevent their absorption through the intestinal wall and into the bloodstream. The microflora imbalance resulting from colonization and aggregation of these bacteria may trigger many conditions that may include colibacillosis, rota virus, corona virus, clostridium perfringens, cryptosporidiosis, coccidosis, and/or septicemia. A wide range of symptoms, such as scours, intestinal lesions, and/or enteritis, may accompany these afflictions, impeding nutrient uptake, decreasing feed intake, and preventing the healthy maturation of intestinal microflora. In light of these conditions and because smaller and/or unhealthy animals are more difficult to adequately manage due to their specific housing, dietary, and husbandry needs, maintaining an intestinal environment conducive to nutrient uptake is critical to the health and development of young ruminant animals.

Work to determine the proper attributes for milk replacers has been conducted during the last several decades. One result of this work is the widespread use of antibiotics to combat harmful bacteria in the intestine, but frequent antibiotic treatment can lead to bacterial resistance. New approaches to ridding the intestinal lumen of bacterial strains detrimental to healthy growth and development in ruminant animals are therefore highly desired.

### SUMMARY

Implementations provide methods of feeding a milk replacer to ruminants that includes an amount of soluble mannan oligosaccharides.

In various implementations and alternatives, the ruminants may increase a rate of weight gain, increase feed efficiency, decrease a rate of milk replacer refusal, and/or increase consumption of a starter feed in response to ingesting the milk replacer.

In various implementations and alternatives, the ruminants may consume about 0.1 to about 6 grams of the soluble mannan oligosaccharides per day.

In additional implementations and alternatives, the ruminants may consume about 0.5 to about 1.5 pounds of milk replacer per day, or about 1.5 to about 3.0 pounds of milk replacer per day.

In additional implementations and alternatives, the milk replacer includes about 18 to about 30 percent by weight of protein in powder form and/or about 15 to about 31 percent by weight of fat.

In addition or alternatively, the ruminants are fed the milk replacer until weaned.

In additional implementations and alternatives, the mannan oligosaccharides are extracted from a yeast cell wall. In such cases, the yeast may be a species selected from a group of fungal genera including Paecilomyces, Saccharomyces, and/or Ganoderma.

In additional implementations and alternatives, the step of feeding the ruminants further involves feeding forage and/or antibiotics to the animals.

In addition or alternatively, the soluble mannan oligosaccharides are phosphorylated.

In another implementation, a method of feeding milk replacer to calves is provided, where the milk replacer includes an amount of soluble mannan oligosaccharides and the calves are between about 0 and about 6 weeks of age. In response to ingesting the milk replacer that includes soluble mannan oligosaccharides, the calves increase a rate of weight gain.

### DETAILED DESCRIPTION

Establishing and maintaining a healthy intestinal microbiome helps prevent gastrointestinal disease and facilitates adequate nutrient uptake. Harmful bacteria present in the intestine may disrupt a healthy, balanced microbiome by accumulating in large numbers. More specifically, pathogenic bacteria often bind the mucosa and/or epithelial cells that comprise the inner intestinal wall by means of small, hair-like projections on their surfaces known as pili or fimbriae. These projections are rich in lectins, which are carbohydrate-binding proteins. Because lectins bind specific carbohydrate molecules, bacteria with mannose-specific pili bind intestinal epithelial cells with mannose molecules at their surface. Attachment of pathogens at the epithelial layer can lead to pathogenic colonization, subsequently causing disease.

By providing a mannose molecule that binds to mannose-specific pathogens, mannan oligosaccharides offer a potential means to combat bacterial colonization and infection by sequestering pathogens that might otherwise attach to the intestinal wall. Mannan oligosaccharides are glucomannoprotein molecules derived from the outer portion of fungal cell walls that are capable of binding mycotoxins. Mannan oligosaccharides are also resistant to enzymatic digestion in the gut, enabling them to pass through the intestinal tract while towing any bacterial cells they are able to bind. Additionally, mannan oligosaccharides may elicit an immune response by stimulating the secretion of mannose-binding proteins from the liver. These proteins bind bacteria in the intestine, which initiates an immune cascade that not only responds to foreign bacteria, but also fortifies the existing immune system. Although their natural affinity for bacterial pathogens makes them an attractive milk replacer additive for ruminant animals, the effectiveness of mannan oligosaccharides to improve animal performance remains limited.

Applicant's discovery of methods for feeding milk replacer products that result in increased animal performance is the surprising result of experimentation with various forms of mannan oligosaccharide supplements. Applicant understood that mannan oligosaccharides were capable of binding harmful bacteria in the intestine of ruminant animals, but the inefficiency of this binding prevented a more robust improvement of intestinal microflora. More specifically, the ability of mannan oligosaccharides to bind bacteria remained physically hindered by extraneous cell wall components not removed during their isolation from fungal cell walls, which are comprised of approximately 30 to 60 percent polysaccharides, about 15 to 30 percent proteins, and about 5 to 20 percent lipids. These cell wall components also inhibited mannan oligosaccharides from acting as a substrate for beneficial bacteria, such as bifidobacteria and lactobacillus, which prevented them from out-competing the pathogenic strains present in the gut. Accordingly, Applicant endeavored to develop a new method of feeding ruminant animals milk replacer products supplemented with more precisely-extracted mannan oligosaccharides that results in improved animal performance. Provided herein are methods of feeding milk replacer products comprised of soluble mannan oligosaccharide products. By readily dispersing within the intestinal tract and agglutinating a greater number of bacteria, while also fostering the growth of favorable microbes, the solubility of the mannan oligosaccharides provided to ruminant animals according to the feeding method herein leads to observed decreases in milk replacer refusal and increases in weight gain, feed efficiency, and starter feed consumption.

While the products and methods of the present disclosure are described in relation to ruminant milk replacer and feeding ruminant animals, such as ruminants prior to and/or during weaning, these products may be produced for other animals and be fed similarly in order to result in improved performance. Other animals may include, but are not limited to: livestock animals, young livestock animals, horses, foals, pigs, piglets, zoo animals, young zoo animals, companion animals, and young companion animals. These animals may ingest such milk replacers through weaning, which may vary between species. For instance, young ruminants may be weaned after about 12 weeks from birth, sometimes less. In some implementations, the animals may ingest milk replacers at any stage in life, for instance, when the animal requires a liquid diet or a semi-liquid diet rich in nutrients that simultaneously combats pathogenic colonization.

Milk Replacer Compositions Containing Soluble Mannan Oligosaccharides

Milk replacers of the present disclosure may include or be admixed with soluble mannan oligosaccharides.

Sources of mannan oligosaccharides may include, but are not limited to: eukaryotic cell walls derived from species of fungi that may belong to the Paecilomyces, Saccharomyces, and/or Ganoderma genus. In one embodiment, eukaryotic cells walls are derived from a single species of fungus. In a preferred embodiment, eukaryotic cell walls are derived from Saccharomyces cerevisiae. In other embodiments, eukaryotic cell walls are derived from two or more fungal species. The incorporation of cell walls derived from multiple fungal species in a single composition may result in a synergistic effect by increasing the diversity of bacterial binding sites present on the mannan oligosaccharides.

The mannan oligosaccharides used according to the methods disclosed herein are soluble. As used herein, the term “soluble” means that the mannan oligosaccharides may be totally or at least partially dissolved in water. The solubility of the mannan oligosaccharides used herein results from their enhanced differentiation and purification from the fungal cell walls used to derive them. The concentration of soluble mannan oligosaccharides may be greater relative to preexisting feed additive products. In some embodiments, a totally-soluble mannan oligosaccharide composition may be used. Totally-soluble compositions may be 100 percent soluble and may be produced by isolating pure mannan oligosaccharide, which is free of, or with substantially reduced levels of, unwanted fungal cell wall components such as β-glucans, glucose, protein, nitrogen, and/or fat. In some embodiments a partially-soluble mannan oligosaccharide composition may be used. Partially-soluble compositions may be produced by mixing totally-soluble mannan oligosaccharides with insoluble mannan oligosaccharides. Alternatively, partially-soluble mannan oligosaccharides may be produced by manufacturing processes that partially separate the mannan oligosaccharides from the cell wall. Partially-soluble compositions may be about 30 to about 99 percent, about 50 to about 90 percent, about 55 to about 80 percent, or about 58 to about 70 percent soluble in water.

The mannan oligosaccharide content of the milk replacer may vary and may be adjusted according to the needs and/or condition of the animal. Levels may also vary depending on the solubility desired, e.g., whether totally-soluble or partially-soluble mannan oligosaccharide compositions are desired. In some embodiments, the soluble (e.g., totally-soluble or partially-soluble) mannan oligosaccharides present within the milk replacer may comprise about 0.01 to about 1.50 percent, about 0.01 to about 1.0 percent, about 0.1 to about 1.0 percent, or about 0.1 to about 0.9 percent by weight of the dry milk replacer in powder form. For example, about 2.5 grams of soluble mannan oligosaccharides may be included in a 1.5-pound daily ration of dry milk replacer powder. (1.50 lbs.=680.389 grams→2.5 grams soluble mannan oligosaccharides/680.389 grams milk replacer powder=0.367% soluble mannan oligosaccharide content).

Soluble mannan oligosaccharide additives may be provided in powder form. The powder may comprise various amounts of soluble mannan oligosaccharides depending on the process used to derive them. In one embodiment, partially-soluble mannan oligosaccharides may be provided as a concentrated yeast cell wall product. Partially-soluble mannan oligosaccharides may be present in a concentrated yeast cell wall product at about 50 to about 80 percent, about 55 to about 70 percent, about 60 to about 66 percent, or about 62 to about 66 percent by weight of the concentrated yeast cell wall product, with the remaining portion of the product composed of fungal cell wall constituents. The remaining fungal cell wall constituents may include, but are not limited to: glucose, nitrogen, protein, ash, fat, and β-glucan molecules. In another embodiment, a totally-soluble mannan oligosaccharide extract may be provided. Totally-soluble mannan oligosaccharides may be present in a concentrated extract at about 60 to about 95 percent, about 70 to about 90 percent, or about 80 to about 88 percent by weight of the concentrated mannan oligosaccharide extract, with the remaining portion of the concentrate composed of yeast cell wall constituents.

In some embodiments, the soluble mannan oligosaccharides may be phosphorylated. Addition of one or multiple phosphate groups to each mannan oligosaccharide molecule may improve its bacterial binding capacity.

Milk replacers of the present disclosure may be produced according to traditional methods in which the fat and protein components of milk replacers are spray dried and combined into a milk replacer powder. Spray drying processes generally involve maintaining a spray dryer at temperatures between 100° C. to 200° C. so that the spray dried component rapidly heats and loses moisture. Following spray drying, the spray dried powder is subjected to a subsequent heating step, such as in a dryer drum, with an air temperature of between 100° C. to 200° C. in order to further reduce the moisture content of the powder.

The nutrient profile of the milk replacer generally includes fat and protein. The fat content may range from about 2.25 to about 4.7 weight percent of the hydrated milk replacer or from about 15 to about 31 weight percent of the milk replacer powder. Predominant fat sources may be lard, tallow, palm kernel or coconut oils, alone or in combination. In addition, some fat from lecithin and residual fat (e.g., butter fat, milkfat or both) may contribute to the fat content in milk replacers. The level of fat may be tailored for a target animal, and for instance, calf milk replacers may have the aforementioned fat content of between about 15 and about 31 weight percent of the powder. In a more particular example, traditional calf milk replacers may include fat from about 20 to about 25 weight percent of the powder or about 3 to about 3.75 weight percent of the hydrated milk replacer, and full potential calk milk replacers may include fat from about 25 to about 31 weight percent of the powder or about 3.75 to about 4.7 weight percent of the hydrated milk replacer.

Protein in milk replacers typically ranges from about 2.2 to about 5.1 weight percent of the hydrated milk replacer or about 18 to about 30 weight percent of the powder. Protein may be sourced from animal (e.g., milk, plasma, egg and red blood cells) and vegetable sources and combinations thereof. In some implementations, the protein in milk replacer may be all milk protein. Milk-derived protein sources are generally referred to as milk proteins and may include whey, casein, skim milk, sodium caseinate and calcium caseinate. Alternatively, non-milk proteins, such as vegetable proteins (e.g., soy protein), animal protein (e.g., plasma, egg and red blood cells), and single cell proteins, may be included as a protein source in the milk replacer. Non-milk protein may account for up to from 0 to about 65 percent, from about 50 to about 65 percent, or up to or at about 50 or about 65 percent of the total protein content, with the balance of protein derived from milk protein; meaning milk protein may account for 100 percent, about 1 to 35 percent, about 35 to about 50 percent, up to about 50 percent, or about 35 percent of the total protein content in the milk replacer. For traditional calf milk replacers, the protein content may be about 22 weight percent of the powder or about 3.3 weight percent of the rehydrated milk replacer, and milk replacers formulated for enhanced performance, such as full potential milk replacers, may include protein at about 26 to about 28 percent of the powder or about 3.9 weight percent to about 4.8 weight percent of the rehydrated milk replacer.

Methods of Feeding Milk Replacers Containing Soluble Mannan Oligosaccharides

Methods of feeding animals may involve feeding the animals milk replacer containing soluble mannan oligosaccharides. This approach may include obtaining a soluble mannan oligosaccharide product and combining it with a milk replacer just prior to feeding. Alternatively, the milk replacer may contain the soluble mannan oligosaccharide.

Generally, animals are offered a fixed amount of milk replacer per day, which may form all or a portion of the young animal's daily feed ration. Prior to the onset of weaning, the milk replacer in the feed ration may be offered twice per day, and may generally be divided into equal parts.

Milk replacers may be fed in traditional settings at a rate of about 1.25 pounds per day on a dry weight basis during the first week of life. Thereafter, the animal may be offered about 1.5 pounds of milk replacer per head per day on a dry weight basis. At the onset of weaning, the animal may be offered one feeding per day, totaling about 0.75 pounds of milk replacer per head per day on a dry weight basis.

In enhanced feed settings, full potential milk replacers may be fed at a rate of at least about 1.6 pounds up to about 3.0 pounds per head per day on a dry weight basis. For instance, in the first week of life, young animals, such as calves, in a full potential setting may be offered about 1.6 pounds or more (e.g., up to about 1.9 pounds) of milk replacer per head per day on a dry weight basis. From the second week of life onward, such animals in a full potential setting may be offered the same amount (about 1.6 pounds) of milk replacer or may be offered up to 3.0 pounds of milk replacer per head per day on a dry weight basis. Thereafter, the amount of milk replacer offered to the young animal may be maintained or the level may decrease, for example, depending on the timing of the onset of weaning.

Prior to feeding the animals, the milk replacers are hydrated with water or another aqueous material. In one embodiment, an amount of soluble mannan oligosaccharide additive may be admixed with the rehydrated milk replacer. Alternatively, the soluble mannan oligosaccharides may be added to the milk replacers prior to rehydration, and under these circumstances, it may be preferable to provide the soluble mannan oligosaccharides in solid form.

The amount of soluble mannan oligosaccharides fed to and available for consumption by the ruminant animals is based on the animal's size, health status, age, and activity level. For instance, the amount of mannan oligosaccharides may be increased if the animal exhibits symptoms of gastrointestinal disease. Alternatively, the amount of mannan oligosaccharides may be decreased as the animal ages and develops a mature intestinal microflora. In other embodiments, a young animal may be fed about 0.1 to about 6 grams, about 0.1 to about 5 grams, or about 1 to about 4 grams, or about 2 to about 4 grams of soluble mannan oligosaccharide per day. In other embodiments, a mature animal may be fed about 5 to about 25 grams, about 7 to about 23 grams, or about 10 to about 20 grams of soluble mannan oligosaccharides per day.

In some embodiments, one or more antibiotics may also be provided to the animals concurrently with the milk replacer comprising soluble mannan oligosaccharides. Antibiotics may be employed in response to animals exhibiting acute disease symptoms. Antibiotics may also be provided in response to the detection of specific strains of bacteria present within an animal's digestive tract.

In addition to milk replacer, starter feed may be offered to the young animals on an ad libitum basis. Starter feeds, such as calf starter feeds, may include a mixture of one or more of corn, soybean meal, wheat middlings, oats, molasses, fat, ground cotton seed hulls, distillers grains, calcium carbonate, salt, and macronutrients and micronutrients. The starter feed may contain about 45 to 50 percent coarse ingredients such as corn, soy and oats; about 16-22 percent protein; about 2 to 3 percent fat; about 5 to 6 percent fiber (determined on a NIR basis); about 7 percent acid detergent fiber; about 6 percent molasses; and the balance including a mixture of other nutrients. The amount of starter feed offered to the young animals may increase as the animals progress through the weaning process.

In addition to milk replacer, forage may be provided to the young animals to promote optimal digestive health. Sources of forage may include grasses, long-stem hay, hay cubes, and hay pellets. The amount of forage offered or provided to the young animals may increase as the animals progress through weaning.

Ingestion of milk replacers containing soluble mannan oligosaccharides by young animals according to the present disclosure has been found to improve performance. For instance, young animals may increase weight gain, may increase intake of starter feed, may reduce feed refusal of milk replacer, and/or may decrease a feed-to-gain ratio such that less feed is consumed to increase weight gain. Improved performance may result in reduction of the age of freshening or the onset of lactation, thereby reducing the cost of milk production; or may cause the animal to increase weight gain from early age, which may reduce the cost of beef production.

In various embodiments, ingestion of soluble mannan oligosaccharides may result in sequestration of various species of bacteria, which may include but are not limited to: Escherichia coli, Staphylococcus aureus, and/or various species of Pseudomonas, Salmonella, Shigella, and Vibrio. When bound by soluble mannan oligosaccharides, these bacteria cannot multiply and are starved to death.

Implementations of the present disclosure are more particularly described in the following calf trials that are for illustrative purposes only. Numerous modifications and variations are within the scope of the present disclosure as will be apparent to those skilled in the art.

### EXAMPLES

Calf Trial 1

This study was conducted to assess the ability of soluble mannan oligosaccharide products to increase calf performance. Measured performance parameters included weight gain, milk replacer consumption, and starter feed consumption. By measuring the effects of mannan oligosaccharides at a range of solubility levels, this study revealed the significant improvement in gastrointestinal health that may be achieved by feeding calves according to the methods disclosed herein.

Eighty bull calves, each between two and five days old, were sourced from Wisconsin and shipped to Eastern Missouri, where they were treated according to standard receiving protocol. The following day, the calves were assigned to experimental treatments and the tests initiated.

Sixteen calves were allotted to each of five treatments where each was offered the same conventional milk replacer (Land O' Lakes Maxi-Care Plus), which contains 22 percent protein and 20 percent fat. Treatments for the two-week trial included: no mannan oligosaccharides (negative control); BioMos (conventional control #1) with insoluble mannan oligosaccharides; Fulfill (conventional control #2) with insoluble mannan oligosaccharides; partially-soluble mannan oligosaccharides; or totally-soluble mannan oligosaccharides. BioMos was sourced from Alltech, Inc., Nicholasville, Ky., Fulfill was sourced from Safmannan, Inc., Quincy, Ill., and both the solubility enhanced and totally soluble products were sourced from Biothera, Inc., Eagan, Minn. Calves were fed 0.75 lbs. milk replacer twice daily (1.50 lbs./day) for the duration of the trial. The milk replacer powder was reconstituted with 110° F. water. The calves were also fed a 20 percent crude protein starter, ad libitum, (Purina AmpliCalf 20) throughout the trial.

The conventional mannan oligosaccharide products used in the trial, BioMos and Fulfill, were collected and assayed for mannoprotein content prior to initiation. Both products contained 46 percent mannoprotein and no soluble mannoprotein. Pursuant to the feeding recommendations for the BioMos product, BioMos and Fulfill were fed at a rate of 4 grams per head per day. The partially-soluble and totally-soluble products contained 65 percent and 86 percent mannoprotein, respectively. Inclusion of these products was reduced so that all milk replacer diets provided iso-levels of mannoprotein. Therefore, the partially-soluble and totally-soluble mannan oligosaccharide products were fed to provide 2.83 or 2.13 grams per head per day. The partially-soluble mannan oligosaccharide treatment was produced by adding totally-soluble mannan oligosaccharides to an insoluble mannan oligosaccharide product.

Table 1 lists performance data for calves fed different sources of mannan oligosaccharides.

TABLE 1
Performance of calves fed control milk replacer and milk
replacer supplemented with conventional, partially-soluble,
and totally-soluble mannan oligosaccharide products

| Treat- | Negative | | | Partially | Totally | |
|---|---|---|---|---|---|---|
| ment | Control | BioMos | Fulfill | Soluble | Soluble | SE |
| Initial | 3.14 | 3.19 | 3.36 | 3.47 | 3.27 | 0.31 |
| Ig | | | | | | |
| Initial | 97.8 | 97.2 | 97.1 | 97.5 | 97.2 | 1.19 |
| Weight, | | | | | | |
| lbs. | | | | | | |
| Final | 104.6 | 104.4 | 104.0 | 106.7 | 107.2 | 1.51 |
| Weight, | | | | | | |
| lbs. | | | | | | |

Average Period Gain, lbs.

| | | | | | | |
|---|---|---|---|---|---|---|
| Week 1 | 3.24ab | 3.43ab | 1.94a | 4.16ab | 4.97b | 0.86 |
| Week 2 | 3.65 | 3.79 | 4.93 | 5.01 | 5.11 | 0.87 |
| Total | 6.80a | 7.21ab | 6.87a | 9.17ab | 10.08b | 1.09 |
| Gain | | | | | | |

Average Period Milk Replacer Consumption, lbs. (DM Basis)

| | | | | | | |
|---|---|---|---|---|---|---|
| Week 1 | 8.94a | 9.96c | 9.00ab | 9.74bc | 10.16c | 0.26 |
| Week 2 | 9.87a | 10.17ab | 9.95a | 10.25ab | 10.47b | 0.17 |

Total Calf Milk Replacer Consumption

| | | | | | |
|---|---|---|---|---|---|
| 18.82a | 20.14c | 18.96ab | 19.99bc | 20.63c | 0.37 |

Period Starter Consumption, lbs. (DM Basis)

| | | | | | | |
|---|---|---|---|---|---|---|
| Week 1 | 0.17 | 0.21 | 0.19 | 0.26 | 0.20 | 0.08 |
| Week 2 | 0.70 | 0.75 | 0.68 | 1.33 | 1.06 | 0.25 |

Total Starter Consumption, lbs. (DM Basis)

| | | | | | |
|---|---|---|---|---|---|
| 0.87 | 0.96 | 0.87 | 1.59 | 1.26 | 0.31 |

Average Feed: Gain

| | | | | | |
|---|---|---|---|---|---|
| 5.57ab | 3.56ab | 6.91b | 2.85ab | 2.46a | 1.53 |

Statistical p values represent overall ANOVA values. Averages in the same row not followed by a common letter differ (p < 0.05) using LSD procedure.

The data from Table 1 confirm that the total two week gain for calves fed mannan oligosaccharide products was generally improved compared to calves not fed mannan oligosaccharides. Compared to the negative control, average two week weight gain of the conventional mannan oligosaccharide products was increased by 3.5 percent while that for the products containing partially-soluble mannan oligosaccharides was improved 41.5 percent. The improvement in average weight gain was even greater for totally-soluble mannan oligosaccharide products compared to the negative control (48.2 percent). The calves fed totally-soluble mannan oligosaccharides also gained significantly (p<0.05) more weight than the Fulfill animals. Furthermore, calves fed the totally-soluble treatment refused less milk replacer than calves assigned to the negative control or Fulfill groups (p<0.05). Feed efficiency was also significantly improved in the totally-soluble treatment relative to the Fulfill treatment (p<0.05).

To better understand the impact of soluble mannan oligosaccharides on calf performance, the data for calves fed conventional mannan oligosaccharide products containing small amounts of soluble mannan oligosaccharides were pooled and compared to the data for calves receiving high levels of soluble mannan oligosaccharides (partially-soluble+totally-soluble). Table 2 lists the performance values for this pooled data.

TABLE 2
Performance of calves fed conventional mannan
oligosaccharide products vs. calves fed mannan
oligosaccharides with enhanced solubility

| | Conventional | Soluble | |
|---|---|---|---|
| MOS Type | (Treatments 2 + 3) | (Treatments 4 + 5) | SE |
| Initial Ig | 3.27 | 3.37 | 0.22 |
| Initial Weight, lbs. | 97.2 | 97.3 | 0.82 |
| Final Weight, lbs. | 104.2 | 107.0 | 1.03 |

Average Period Gain, lbs.

| | | | |
|---|---|---|---|
| Week 1 | 2.73a | 4.57b | 0.59 |
| Week 2 | 4.32 | 5.06 | 0.63 |
| Total Gain | 7.05a | 9.63b | 0.75 |

Average Period Milk Replacer Consumption, lbs. (DM Basis)

| | | | |
|---|---|---|---|
| Week 1 | 9.52 | 9.95 | 0.17 |
| Week 2 | 10.07a | 10.36b | 0.10 |
| Total CMR | 19.59a | 20.31b | 0.23 |

Period Starter Consumption, lbs. (DM Basis)

| | | | |
|---|---|---|---|
| Week 1 | 0.20 | 0.23 | 0.05 |
| Week 2 | 0.72 | 1.19 | 0.19 |
| Total Dry Feed Cons. | 0.92 | 1.42 | 0.23 |
| Average Feed: Gain | 5.12c | 2.66d | 0.90 |

abaverages in the same row not followed by a common letter differ (p < 0.05) using LSD procedure.
cdaverages in the same row not followed by a common letter differ (p < 0.06) using LSD procedure.

The pooled data in Table 2 strongly suggests that products containing soluble mannan oligosaccharides are superior to conventional mannan oligosaccharide products. Total calf gain was improved by over 36 percent (p<0.05) in soluble mannan oligosaccharide treatment animals. Milk replacer refusal was also significantly reduced (p<0.05). The amount of feed required per unit of gain by calves fed the soluble products was reduced by nearly half (p<0.06).

As used herein, the term “about” modifying, for example, the quantity of a component in a composition, concentration, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

Similarly, it should be appreciated that in the foregoing description of example embodiments, various features are sometimes grouped together in a single embodiment for the purpose of streamlining the disclosure and aiding in the understanding of one or more of the various aspects. These methods of disclosure, however, are not to be interpreted as reflecting an intention that the claims require more features than are expressly recited in each claim. Rather, as the following claims reflect, inventive aspects lie in less than all features of a single foregoing disclosed embodiment, and each embodiment described herein may contain more than one inventive feature.

Although the present disclosure provides references to preferred embodiments, persons skilled in the art will recognize that changes may be made in form and detail without departing from the spirit and scope of the invention.

What is claimed is:

1. A method of feeding ruminants, the method comprising:
feeding the ruminants a milk replacer, the milk replacer comprising an amount of mannan oligosaccharides,
wherein the mannan oligosaccharides are soluble.

2. The method of claim 1, wherein in response to ingesting the milk replacer, the ruminants increase a rate of weight gain.

3. The method of claim 1, wherein in response to ingesting the milk replacer, the ruminants increase feed efficiency.

4. The method of claim 1, wherein in response to ingesting the milk replacer, the ruminants decrease a rate of milk replacer refusal.

5. The method of claim 1, wherein in response to ingesting the milk replacer, the ruminants increase consumption of a starter feed.

6. The method of claim 1, wherein the ruminants consume between about 0.1 and about 6 grams of the soluble mannan oligosaccharides per day.

7. The method of claim 1, wherein the ruminants consume about 0.5 to about 1.5 pounds of milk replacer per day.

8. The method of claim 1, wherein the ruminants consume about 1.5 to about 3.0 pounds of milk replacer per day.

9. The method of claim 1, wherein the milk replacer includes about 18 to about 30 wt % protein in powder form.

10. The method of claim 1, wherein the milk replacer includes about 15 to about 31 wt % fat in powder form.

11. The method of claim 1, wherein the ruminants are fed the milk replacer until weaned.

12. The method of claim 1, wherein the mannan oligosaccharides are extracted from a yeast cell wall.

13. The method of claim 12, wherein the yeast is a species selected from a group of fungal genera including Paecilomyces, Saccharomyces, and/or Ganoderma.

14. The method of claim 1, wherein the step of feeding the ruminants a milk replacer further comprises feeding forage to the animals.

15. The method of claim 1, wherein the step of feeding the ruminants a milk replacer further comprises feeding antibiotics to the animals.

16. The method of claim 1, wherein the mannan oligosaccharides are phosphorylated.

17. A method of feeding a milk replacer to calves, the milk replacer comprising an amount of soluble mannan oligosaccharides,
wherein the calves are between about 0 and about 6 weeks of age, and
wherein the calves increase a rate of weight gain in response to ingestion of the milk replacer.
